(19) United States
(12) Patent Application Publication
(10) Pub. No.: US 2011/0208036 A1
(43) Pub. Date: Aug. 25, 2011

(54) COUPLED ANTENNA IMPEDANCE SPECTROSCOPY

(75) Inventors: Noel Axelrod, Bnei Brak (IL); Alex Konevsky, Bnei Brak (IL)

(73) Assignee: Physical Logic AG, Zug (CH)

(21) Appl. No.: 13/127,714

(22) PCT Filed: Nov. 5, 2009
(86) PCT No.: PCT/IB09/54914

§ 371 Date: May 5, 2011

Related U.S. Application Data
(60) Provisional application No. 61/111,795, filed on Nov. 6, 2008.

(51) Int. Cl. A61B 5/055; G01R 27/04

(52) U.S. Cl. 600/407; 324/629

(57) ABSTRACT

It has been found advantageous to deploy coiled antennas as transmitters and receivers for acquiring the dielectric spectrum of materials. This method of impendence spectroscopy has been used to determine the concentration of glucose and other small polar molecules in vitro, as well as in vivo by placement on the antennas so that transmission is through the tissue, as for example on opposite sides of an organ or body part. The optimum selection of antenna coils permits deeper penetration into tissue for glucose detection, improves the SNR as well as expands the spectral range for greater accuracy and precision, to enable continuous non-invasive monitoring for either improved patient or automated management of diabetes.

### CROSS REFERENCE TO RELATED APPLICATIONS

This application claims priority to the U.S. Provisional Patent Application having the title “Non Invasive Glucometry Method and Apparatus”, filed on Nov. 6, 2008, having application Ser. No. 61/111,795, which is incorporated herein by reference.

### BACKGROUND OF INVENTION

The present invention relates to the molecular spectroscopy of matter, and more particular the spectroscopy of fluid or tissues in which essentially continuous monitoring can occur without physical sampling, which is removal, of a portion of the fluid or tissue. Even more particularly, the invention relates to the molecular spectroscopy of living tissue for the purpose of determining the concentration of glucose and other small molecules therein.

Prior methods of dielectric or RF spectroscopy have shown correlations between the acquired signals and the blood glucose concentrations.

However, these prior methods suffer a number of recognized deficiencies, in particular electrode polarization, which leads to a loss in signal to noise ratio and other compromises in performance that greatly affect the commercial viability of the methods. Further, such methods appear to measure only the electrolyte imbalances in skin tissue that results from hypo or hyperglycemic events.

Accordingly, it is a first object of the invention to overcome the deficiencies of the prior art methods to provides a non invasive means for blood glucose measurement with a higher signal to noise ratio.

It is a further object of the invention to provide a means for more direct measurement of glucose in tissue that is deeper than the skin and therefore more representative of the availability of glucose at cell membranes.

It is a further object of the invention that the means for direct measurement of glucose in tissue is non-invasive and continuous.

It is a further object of the invention to that this means for more non-invasive and continuous direct measurement of glucose in tissue provides for sufficiently deep penetration to be tissue selective.

It is a further object of the invention that the means for direct measurement of glucose in tissue is not dependent on skin contact reproducibility

6. Higher SNR and wider spectral range for glucose and other molecules of interest

### SUMMARY OF INVENTION

In the present invention, the first object is achieved by providing a process for molecular spectroscopy of a media to determine the concentration of at least one molecular species therein, the process comprising the steps of providing a pair of coiled antennas as electrodes for dielectric spectroscopy measurements, placing the pair of coiled antenna in signal communication through the media, powering at least one of coiled antennas at a first frequency, scanning a frequency range during said step of powering from the first frequency to at least a second frequency, the difference between the first and second frequency representing a first frequency range, acquiring one or more signals from at least one of the coiled antennas during said step of scanning to determine the value thereof, integrating the value of the one or more signals in said step of acquiring, the integration occurring over at least a portion of the first frequency range, calculating the concentration of the molecular species from the integrated value of the one or more signals.

Other objects of the invention are achieved by providing s device for the in-vivo molecular spectroscopy, the device comprising at least one pair of coiled antennas and configured for placement in signal communication with the other antennas in the pair through a first dielectric medium comprising at least a portion of a living organism, a variable frequency power generator in signal communication to each of the antennas in said pair, a signal detector in communication to each of the antennas in said pair for collecting transmitted and reflected signals between each of the antennas over the generated frequency range, a computation means to determine a plurality of signal propagation constants from the detected signals and calculate the concentration of at least one molecular species there from, wherein the pair of coiled antennas have a first resonance below about 100 MHz and the concentration of the molecular species is calculated by integration of one or more of the plurality of signal propagation constants over a frequency range from a first lower frequency to a second upper frequency wherein the second upper frequency is less than about 1 GHz.

Another object of the invention is achieved by providing a process for to calibrate a device for molecular spectroscopy of a media to determine the concentration of at least one molecular species therein, the process comprising the steps of providing at least one sample media through which a plurality of different concentrations of the molecular species is at least one of known and determinable by independent means of the molecular spectroscopy process, providing a pair of coiled antennas as electrodes for dielectric spectroscopy measurements, placing the pair of coiled antennas in signal communication through the sample media, powering at least one of coiled antennas at a first frequency, scanning a frequency range during said step of powering from the first frequency to at least a second frequency, the difference between the first and second frequency representing a first frequency range, repeating said step of scanning of the sample media at plurality of times each corresponding to the different concentrations of the molecular species that is at least one of known and determinable by independent means of the molecular spectroscopy process, acquiring one or more signals from at least one of the coiled antennas during said steps of repeated scanning to determine the value of a plurality of signal propagation parameters, calculating a first correlation product of each of the signal propagation parameters with at least a first subset of the known or determined concentrations of the molecular species, calculating at second correlation product of each of the signal propagation parameters with at least a second subset of the known or determined concentrations of the molecular species, the second subset being larger than the first subset, comparing the first and second correlation products over the first frequency range, identify at least one signal propagation parameter having a selecting regions within the first frequency range wherein the absolute value of the correlation product is greater than about 0.75 over a continuous second frequency range having a width of at least about 50 MHz, calculating the integrated value of each signal propagation parameter identified in the previous step over the continuous second frequency associated therewith with provide at least one Q-band parameters, calculating the correlation of the at least one Q-band parameter to the known or determined concentrations of the molecular species to provide a calibration equation.

The above and other objects, effects, features, and advantages of the present invention will become more apparent from the following description of the embodiments thereof taken in conjunction with the accompanying drawings.

### BRIEF DESCRIPTION OF THE DRAWINGS

FIG. 1 is a block diagram of an apparatus for conducting the inventive method.

FIG. 2A is a plan view of a preferred embodiment of the antennas shown in FIG. 1, whereas FIG. 2B is a fragmented view of an enlarged portion of the antenna in FIG. 2A.

FIG. 3A is a sectional view of a first embodiment of an antenna supporting mold. FIG. 3B is an enlarged orthogonal section through the mold of FIG. 3A. FIG. 3C is an enlarged orthogonal view through the mold of FIGS. 3A and 3B. FIG. 3D is a sectional plan view of another embodiment of an antenna supporting mold.

FIG. 4A is a perspective view of the antenna supporting mold of FIG. 3D, with the test subjects hand inserted showing the external connection to the antenna.

FIG. 4B is a second perspective view of the antenna supporting mold of FIG. 4A with the subject's hand and fingers removed to show the interior pockets.

FIG. 5 is a plot of the calculated electric field penetration of the antenna of FIG. 2 in tissue.

FIG. 6A is a first perspective view from above a more preferred antenna supporting mold that deploys a plurality of antennas on each side of the hand as shown in FIGS. 6A and 6B, whereas FIG. 6B is a second perspective view thereof as seen facing the hand supporting pocket therein.

FIGS. 7A and 7B are plan views of opposite sides of the subjects hand to show the optimum placement of a set of 4 of more preferred generally rectangular antennas.

FIG. 8A cross section elevation through a signal pair of the more preferred antenna of the FIGS. 6 and 7.

FIG. 8B is a plan view of the winding pattern of the coiled antenna of FIG. 8A.

FIG. 8C is a fragmented view of an enlarged portion of the antenna in FIG. 8B.

FIG. 9 is the equivalent circuit used to analyze the results of the frequency scan with the antennas of FIGS. 1 and 2.

FIGS. 10A and 10B compare the spectral response of the S₁₁ and S₁₂ parameters over the frequency spectrum of 300 kHz to 800 MHz. with and without the subject's finger inserted in the antenna supporting mold of FIG. 3.

FIG. 11 is a cross-section elevation of an embodiment of an antenna system that can effectively deploy 2 pairs of coupled electrodes of different length to sample roughly the same projected area of the specimen or tissue.

FIG. 12A is a cross-section elevation of a different embodiment of an antennas that can be deployed with an identical antenna to effective deploy 2 pairs of coupled electrodes of different length to sample roughly the same projected area of the specimen or tissue.

FIG. 12B is a plan view of the coupled antennas in FIG. 12A.

FIG. 13A is a cross-section elevation of a further embodiment of an antennas that can be deployed with an identical antenna to effective deploy 2 pairs of coupled electrodes of different length to sample roughly the same projected area of the specimen or tissue.

FIG. 13B is a plan view of the coupled antennas in FIG. 13A.

FIG. 14 is an example of the function r₂₄ (ω)

FIG. 15 is another antennas transmission spectra of S12 using the antenna configuration in FIGS. 8A and 8B.

FIG. 16 is flowchart illustrating the steps in a process of calibration of the device disclosed herein to non-invasively and continuous monitor blood glucose.

FIG. 17 illustrates an observed correlation of temperature dependence of the integrated intensity of selected model circuit parameters, integrated over specific narrow frequency ranges.

FIG. 18A compares the predicted versus actual blood glucose concentration of a subject using the Q-band parameters in TABLE 1.

FIG. 18B is Clark grid plot of the data such as in FIG. 18A from a plurality of test subjects.

FIG. 19A compares the predicted versus actual blood glucose concentration using the Q-band parameters in TABLE 2.

FIG. 19B is Clark grid plot of the data such as in FIG. 19A from a plurality of test subjects.

FIG. 20 is flowchart illustrating the steps of using the device disclosed herein to non-invasively and continuously monitor blood glucose to after the steps of calibration of FIG. 16.

### DETAILED DESCRIPTION

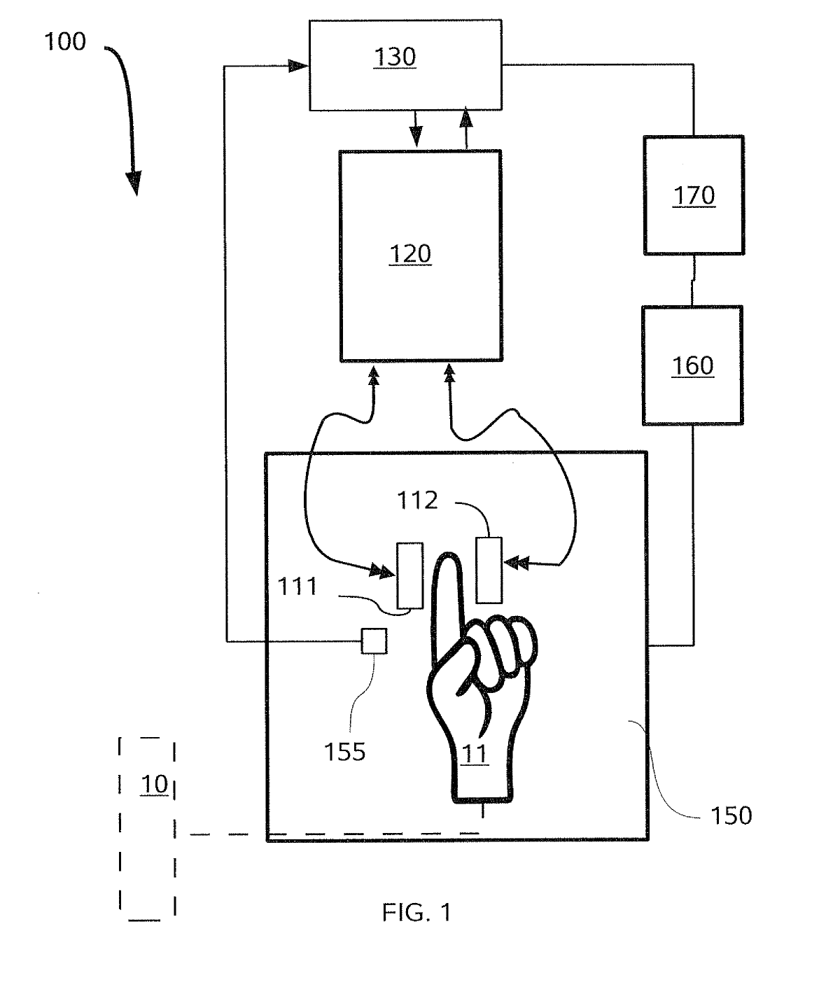

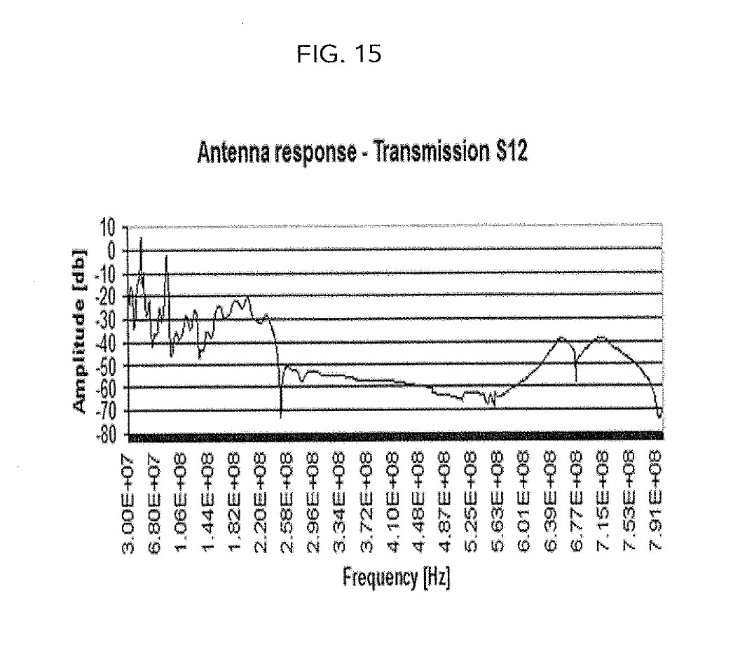

Referring to FIGS. 1 through 20, wherein like reference numerals refer to like components in the various views, there is illustrated therein a new and improved device and method of Coupled Antenna Impedance Spectroscopy.

One embodiment of the inventive apparatus 100 for Coupled Antenna Impedance Spectroscopy is shown in FIG. 1 and can be deployed for either in vivo detection or in vitro samples. Apparatus 100 deploys a pair of coiled or patch antennas 111 and 112 on opposing sides of a test tube 10 (for in vitro measurement) or a limb 11, such as a finger, for in vivo measurements. It should be appreciated that in place of a test tube, a continuously flowing dielectric media can be sampled, such as a pipe in a process stream. The antennas 111 and 112 are energized via a vector network analyzer (VNA) 120. The vector network analyzer 120 is in signal communication with a general purpose computer 130 or microprocessor to perform calculations and calibration processes described in further detail below. The same or a different computer or microprocessor can control the VNA 120. Further, it is also highly preferred that a thermometer 155 or other means can be provided to measure the sample or body temperatures, such as a thermocouple or a non-contact infrared thermometer, which is also in signal communication with the computer or microprocessor 130.

It should be appreciated that high quality cables and connectors should be used to connect the pair of coiled or patch antennas 111 and 112 to the VNA 120 to minimize signal to noise and variability with subject or sample movement.

In initial experiments, the temperature was controlled by placing the antennas 111 and 112 along with the sample in a temperature controlled box or low temperature oven 150, having a fan and heaters (not shown) in signal communication with a relay box 160. The relay box 160 was connected to a control box 170. The control box 170 was in signal communication with the same computer 130 used for control and data acquisition of the VNA 120 signals, as well the temperature measurements from thermocouple 155, placed at or near the skin of limb or finger 11.

The antenna configuration, shown in part in FIGS. 1-4 and 6-8, among others, when used in vivo is preferably deployed non-invasively. Further, the antennas are intended to be energized at a frequency range of about 50 KHz, to 1 GHz, but more preferably from about 200 KHz to 900 KHz. as discussed further below, this results in relatively deep penetration of the electric field, providing what is believed to be a more accurate measurement than prior methods of dielectric spectroscopy, as well as a means for tissue selective measurement of blood glucose. A superior means for the measurement of blood glucose concentrations, and is of great benefit to diabetic patients that require relatively accurate monitoring of blood glucose through the day to manage their food consumption and administration of insulin.

Further, in contrast to prior art methods of dielectric spectroscopy, the method disclosed herein is believed to be capable of providing a higher SNR and wider spectral range for glucose and other molecules of interest.

Prior attempts to measure glucose in the human body by non-invasive dielectric spectroscopy are complicated by two factors the inventive method is believed to overcome. First, the conductivity of biological systems creates electrode polarization with capacitive antennas. The electrode polarization effect results from the accumulation of charge on electrode surfaces and the formation of electrical double layers and can overwhelm the characteristic signal. Various methods have been proposed to correct for this effect, such as are described by Feldman et al.: Time Domain Dielectric Spectroscopy of Biological Systems, IEEE Transactions on Dielectrics and Electrical Insulation Vol. 10, No. 5; October 2003, which is incorporated herein by reference.

Further, according to A. Caduff et al. in “Non-invasive glucose monitoring in patients with diabetes: A novel system based on impedance spectroscopy”, Biosensors and Bioelectronics 22 (2006) 598-604, which is incorporated herein by reference, among others, have noted that dielectric spectroscopy does not measure blood glucose directly, but rather the effect of hyper and hypoglycemic excursions that lead to changes in the electrolyte balance in blood, cells and interstitial fluid (ISF), and is thus an indirect measurement. This occurs in part because the electric field of prior art capacitive sensors only penetrates the skin and the closest underlying tissues to a depth of about 1-2 mm.

In contrast, the inventive technique disclosed herein is believed capable of producing more accurate and reproducible results because it not only avoids electrode polarization, but also probes much deeper tissues.

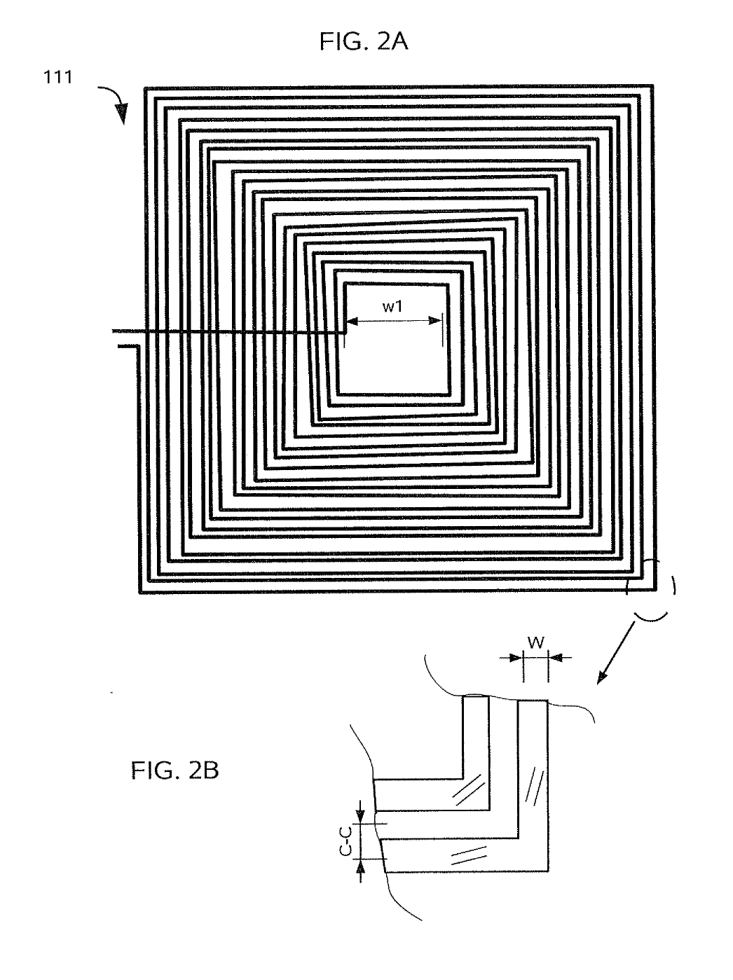

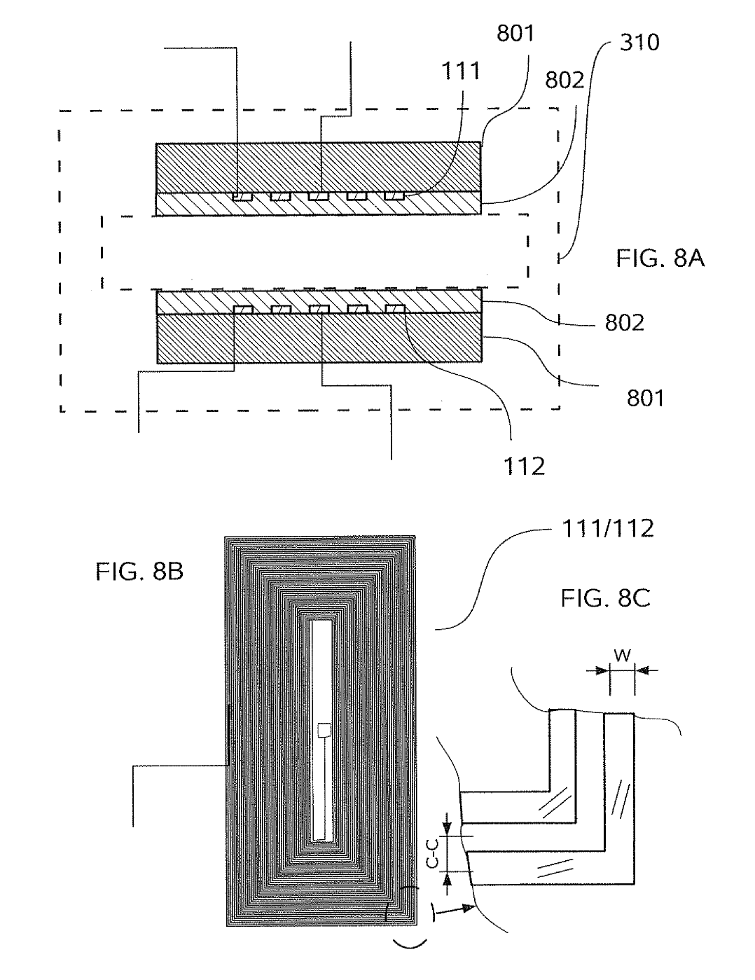

FIG. 2 illustrates in plan view the configuration of the coiled or patch antennas 111 or 112 having a generally spiral configuration. In this spiral configuration there are multiple wraps or winding of a continuous line or conductive stripe around itself in the same plane with at least four or more turns at ends or corners such that the overall shape can be square, rectangular, round, oval or any combination. Further, the topography or shape of the patch antenna deployed herein can be in the form of a loop, coil, spiral or serpentine configuration, as well as combinations of the above. Typically, as illustrated in FIGS. 2A and 2B, the stripe or ribbon portion of the coiled antennas 111 or 112 has a width (W) of about 100 microns, a center to center (C-C) between adjacent lines of about 200 microns and generally at least about of turns so that a section across the entire antenna will bisect about 40 of these lines. The antennas can be printed on general purpose printed circuit boards, or flexible film such as Kapton® and the like, shown as 801 in FIG. 8A. Currently, such antennas are fabricated on a PCB material designated TMMA 10/I available from Rogers Corporation, which has a dielectric constant, ε, of about 10.8 and a minimum thickness of 0.38 mm. As shown in FIG. 2A, for the generally square patch antenna, the wrapping starts around a square with a width (w1) of about 200 microns. Thus the total antenna length is about 70 cm.

The penetration depth of a patch antenna depends both on frequency and antenna configuration. However, for in vivo application penetration depth is primary limited by absorption of electromagnetic radiation by water molecules, and is thus also frequency dependent. Generally, the losses of any given antenna increases as the frequency exceeds 400 MHz, as has been reported in “A 31.5 GHz Patch Antenna Design for Medical Implants”, Ahmed et al., International Journal of Antennas and Propagation, Volume 2008, which is incorporated herein by reference. It has also been reported by Kim et al. “Implanted Antennas Inside a Human Body: Simulations, Designs, and Characterizations”, IEEE TRANSACTIONS ON MICROWAVE THEORY AND TECHNIQUES, VOL. 52, NO. 8, AUGUST 2004, that for a particular antenna energized at 400 MHz, a transmitted communication signals can penetrate 20 cm. Over the frequency range 30 MHz-800 MHz the penetration range corresponding to a loss of 70 dB was in the range of about 5-10 cm. It should be noted that such losses have been of interest to those designing patch antennas for the wireless communication between implanted medical devices and external monitors or control systems.

The penetration range of the antenna 111 and 112 in FIG. 2 have been modeled assuming different properties for underlying tissue, which indicate a useful penetration range of at least about 3-5 cm at the very low frequencies of about 300 KHz to about 400 MHz. Hence, the patch antennas 111 and 112 can be employed on opposite sides of a limb or organ, more directly measure glucose concentrations.

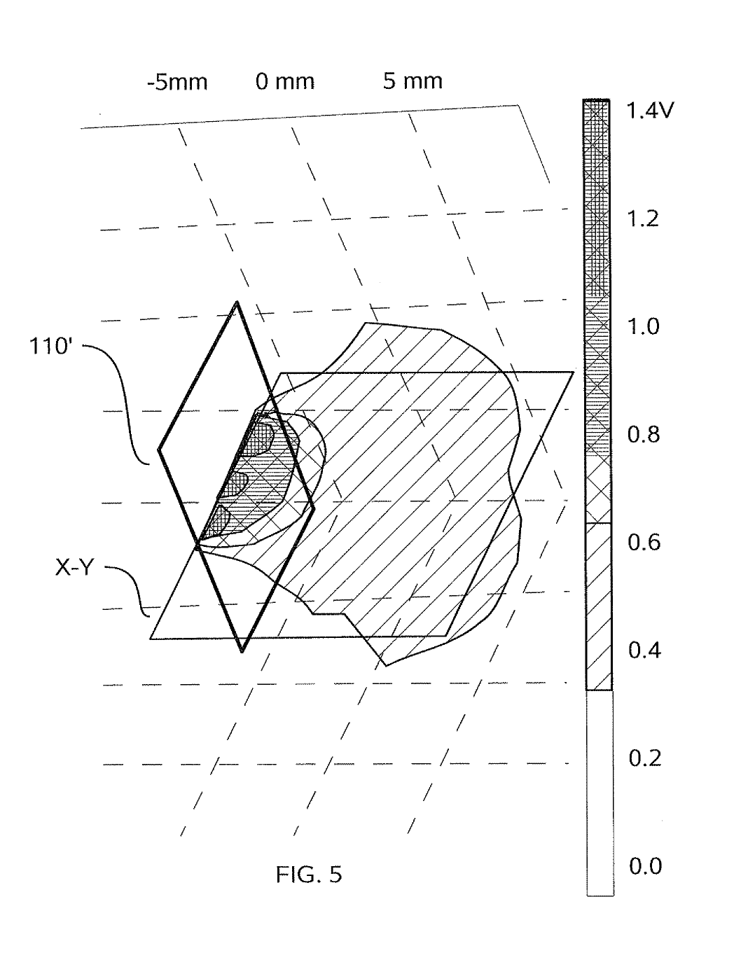

One result of such a simulation of the electromagnetic field penetration within the tissue for the antennas of FIG. 2 is shown in FIG. 5. FIG. 5 is a perspective view of the calculated potential field variation of intensity in the x-y plane is plotted in units of volts, the voltage corresponding to the intensity level of the cross-hatching pattern per the legend bar to the right. The EM field was calculated at 2 MHz. At this frequency the skin dielectric constant was taken εᵣ=900 and conductivity σ=0.12 S/m. In this configuration the patch antenna 111 was connected to the core of the coaxial cable. The dashed lines grid lines are 5 mm apart with the 1 cm wide square electrode being disposed in the x-z plane having the general outer dimensions shown by the rectangle labeled 111′ While the intensity is a maximum of about 1.4V within 3-5 mm from the electrode, the power only drops to about 0.4 V within about 1-2 cm. Thus the general penetration depth of this antenna is in the range 3-5 cm at this very low frequency.

Measurement of glucose are then made by the process of first placing the antennas 111 and 112 on skin, the antennas are then sequentially energized in by the VNA 120 in the frequency scanning mode, with both the transmitted and reflected power measured as the frequency range of each antenna is swept. The frequency sweep speed has an impact on the S/N ratio in the measurements, with the higher speed resulting in a lower is S/N ratio. In the current mode of the operation VNA spectrum sampling rate is about 30 sec. of 800 MHz. During this process raw data are acquired to calculate four signal propagation parameters which vary at least somewhat with frequency for determining the concentration of the molecular species of interest.

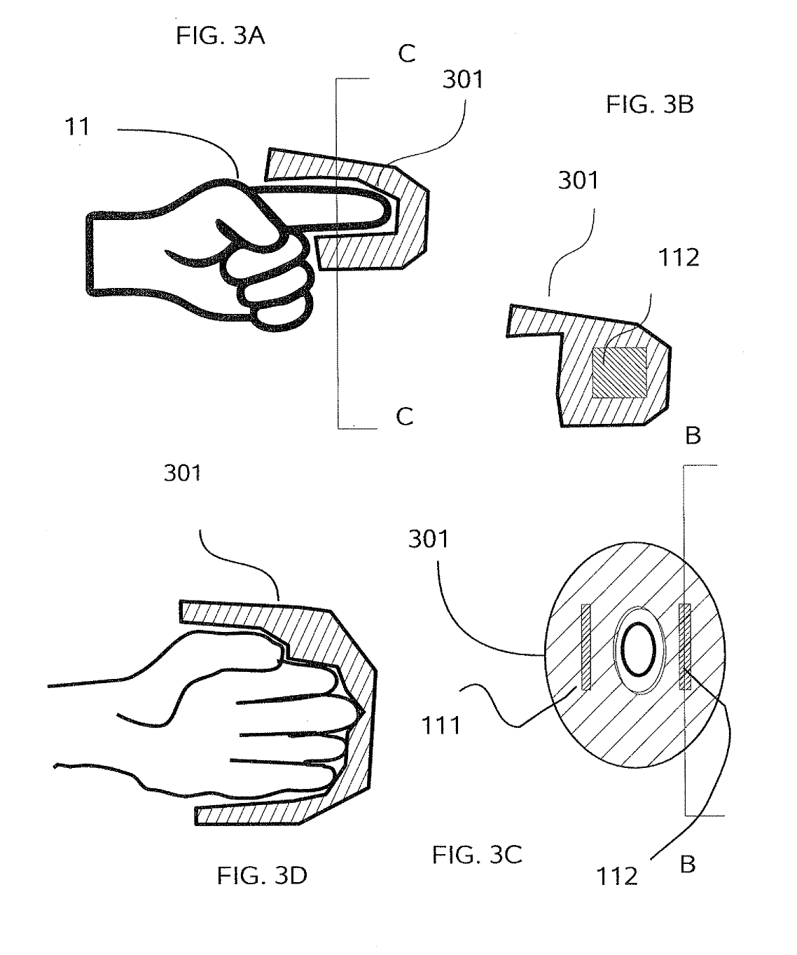

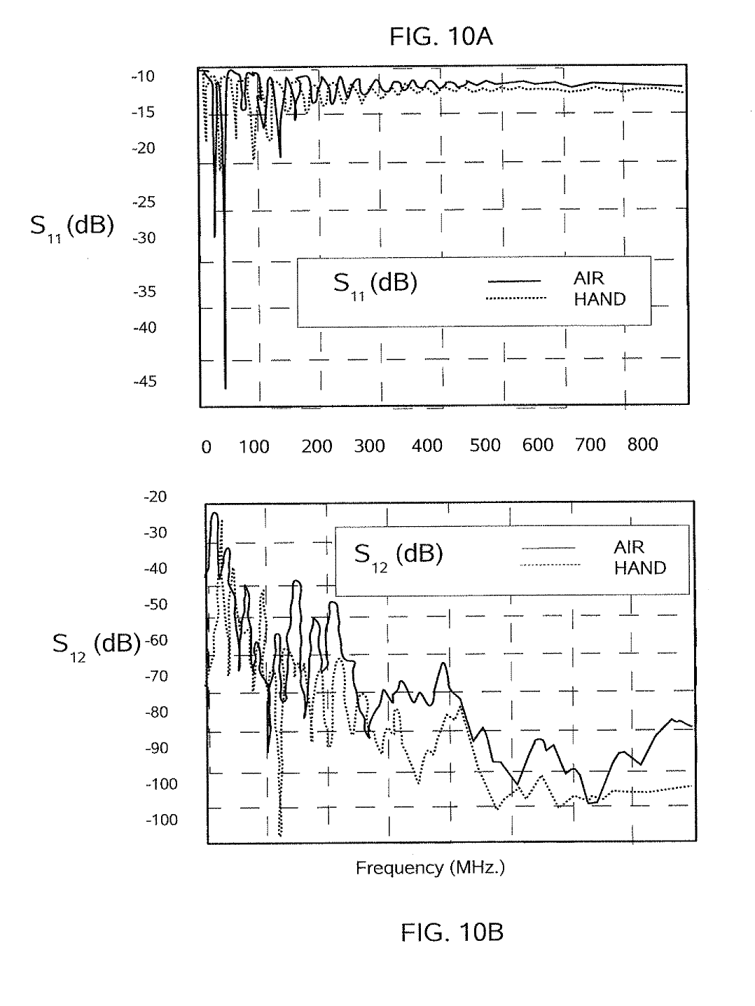

While the patch antenna structure 111 and 112 have a penetration depth and intensity that is highly dependent on its structure, as well as the signal interaction with the dielectric medium being probed, this depth is much greater than the prior art methods, so it is not necessary to place the antennas directly on the skin. Thus, in a more preferred method of using the inventive antenna structure, a molded carrier or support 301 contains and encases the antennas 111 and 112. As the supporting mold 301 is also sculpted or cast to shape of the finger 11, or other appendage, to reproducibly surround the limb or organ portion being probed the placement of the antennas 111 and 112 provides a reproducible spacing from the subject's skin, as the mold 301 fits snugly around the finger. Variations of such antenna supporting molds 301 are shown in FIG. 3A-D, with the actual mold used to generate the experimental data shown in FIG. 10A and FIG. 10B. The antenna supporting mold 301 is optionally made of gypsum or another material that is reasonably transparent, that is having low signal attenuation in the range of about 10 to 900 MHz. It is more preferably to use a plastic cast forming compound, such as ORFIT® Classic, which is available from ORFIT Industries of Wijnegem, Belgium.

In such supporting structure each antennas is wound in a common plane so that antennas in the pair can be placed with their respective common planes parallel and spaced apart. However, depending on the portion of the organism that is sampled, the antennas in the at least one pair can be placed adjacent to each other.

In further contrast to the prior art, it was further discovered that it is undesirable to place the antennas in direct contact with the skin. As the tissue areas with higher electric field have more influence on the S-parameters than with the weaker electric field, electric field for antennas placed on skin is maximum at the skin layer. Therefore, the skin layer may have a dominant influence on S-parameters. The outer skin layer is a source of systematic error for VNA data since it is influenced by the varying environmental conditions such as temperature and humidity. Therefore, it is desirable to reduce its influence on the measurement. One way to do it is to separate antennas from skin by some layer of dielectric material. Another, but less desirable approach includes creation of holder that maintains constant environmental conditions (incubator).

Although the current spacing away from the skin (as shown by the thickness of spacer 802 in FIG. 8A) is by about 1 mm, it is expected that a dielectric spacer with a thickness of 300 μm to about 4-5 mm will be sufficient. The spacing media can be the above cast forming compound from ORFIT, or a comparable dielectric medium.

Thus, spacing of the antennas away from the skin appears to achieve a better correlation between actual blood glucose, such as measured by the YSI method, and then inventive system for several reasons. This is potentially due to the insensitivity to the skin conditions, that is contact, moisture, pressure and the like, but also may reflect representative sampling of the tissue. It is believed that prior methods of dielectric spectroscopy that place the antenna on the skin sample largely the interstitial tissue, while the inventive method is more capable of sampling a larger portion of the arterial and venous blood of the patient/subject.

In one embodiment, the antenna supporting mold 301 in FIG. 3A-C surrounds a single finger, placing a comparably sized antenna 111 and 112 on opposite sides of the finger as shown in the section in FIG. 3C. In contrast, the antenna supporting mold 301 in FIG. 3D, surrounds and immobilizes all the fingers, like a rigid glove, but still disposes the comparably sized antenna 111 and 112 on opposite sides of the finger as shown in the section in FIG. 3C

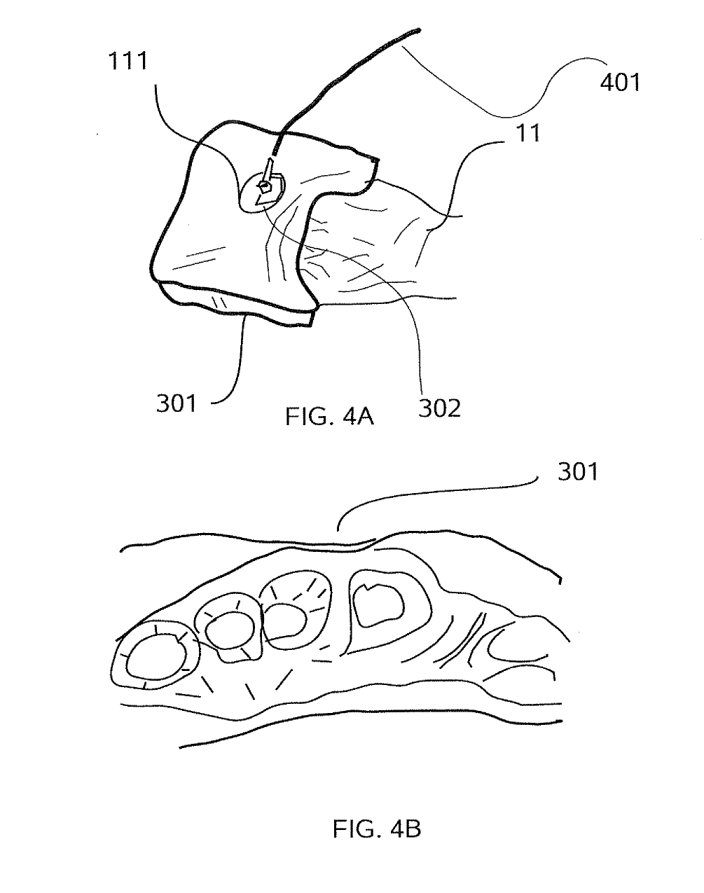

FIG. 4A shows an exterior perspective view the antenna supporting mold 301 of FIG. 3D, which is adapted (as shown in FIG. 4B) to retain each finger in its own pocket it is adapted to receive the entire hand. The cable 401 connects the antenna 111 to the VNA at external connector 302. It is believed that by more completely immobilizing the fingers during measurements the antenna position is less likely to move or creep when the entire hand is in the mold, which will thus improve accuracy and the precision of measurements. Thus, the antenna supporting mold 301 is preferably custom cast for each subject or patient, but may also be provided in a range of generic sizes such as for gloves. Further, the thermocouple 155 may optionally also be encased into the mold and/or deployed at the internal surface of the mold to measure the skin temperature.

It should be appreciated that in addition to the antenna pair being deployed on opposite sides of body portion or appendage, the pair can also be placed adjacent to each other on the same side of the skin or appendage. Accordingly, it is expected that the patch antennas deployed in the inventive method will yield more reproducible and systemic results when properly calibrated for the subject/patient.

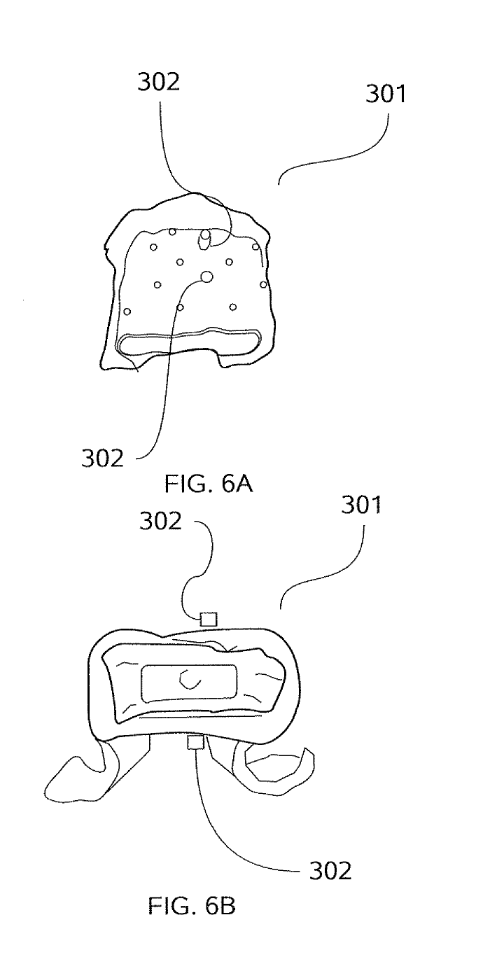

Further, alternative positions or appendages for placement of the antenna are optionally the patient's ear lobe, forearm, wrist, head or leg. In additional it may be preferable to place the inventive antenna system either across the abdominal cavity, as for example to more accurately measure blood glucose within an organ such as the pancreas, as well as on adjacent locations or in closer proximity to larger blood veins or arteries. Thus, for example depending on the body portion used, a particular configuration might be more preferred for patient that desires or requires more continuous monitoring it should be appreciated from the following discussion that the optimum antennas configurations for different portions of the body may be different from what is currently the preferred configuration for making continuous measurement from the hands and finger as illustrated in FIG. 6-8, as for example with respect to the size and number of the antennas deployed. In the embodiment of FIG. 6-8, the supporting mold encased a sufficient portions of the patients/subjects hand to place 2 pairs of antennas in signal communication.

In the frequency scan described above with a single antenna pair the vector network analyzer (VNA) 120 yields four main signal propagation parameters: S₂₂, S₁₁ that represent reflection coefficients and S₂₁, S₁₂ that represent transmission coefficients.

In the models that follow, each S-parameter is a function of time and frequency, where

Sₚ=S(ωᵢ,Tⱼ) (1.1) and
T—time
ω=2πf
f—frequency

The reflection and transmission coefficients Sᵢⱼ can be transformed to four impedance parameters Y₁₁, Y₁₂, Y₂₁, and Y₂₂ by the following formulas:

$$S = [ \begin{matrix}{S}_{11} & {S}_{12} \\ {S}_{21} & {S}_{22}\end{matrix} ] \\ I = [ \begin{matrix}1 & 0 \\ 0 & 1\end{matrix} ] \\ \overline{Y} = \frac{I - S}{I + S} \\ \overline{Y} = \frac{1}{{Z}_{0}} \cdot Y \\ Y = [ \begin{matrix}{Y}_{11} & {Y}_{12} \\ {Y}_{21} & {Y}_{22}\end{matrix} ] \\ Z = {Y}^{- 1} \qquad ( 1.2 )$$

Where Z₀≈50[Ω] is the reference impedance.

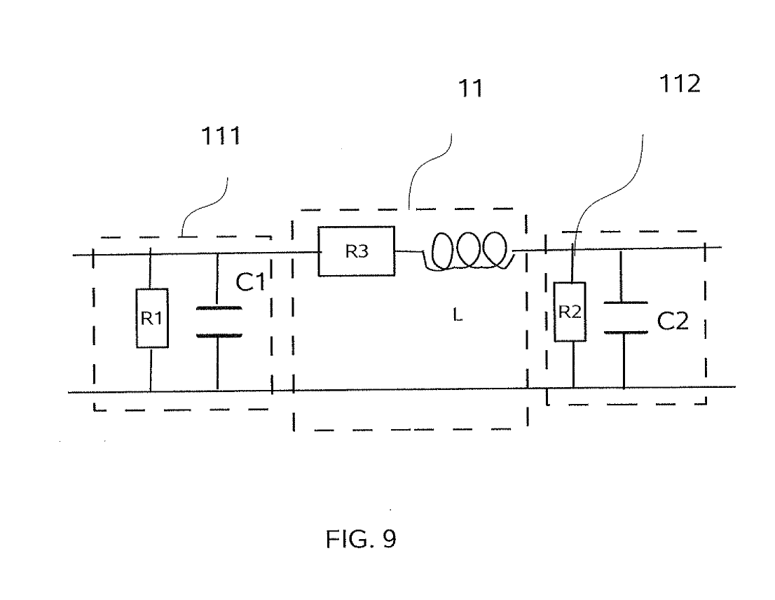

It is possible to model the antennas of FIG. 2 and the intervening sample or dielectric medium by the electric circuit shown in FIG. 9, from which we extract the additional parameters by the following formulas:

$$\begin{matrix}Q = - \frac{Im ( {Y}_{11} )}{Re ( {Y}_{11} )} & ( 1.3 ) \\ {R}_{1} = Re ( \frac{1}{{Y}_{11} + {Y}_{21}} ) & ( 1.4 ) \\ {C}_{1} = Im \frac{{Y}_{11} + {Y}_{21}}{ω} , where ω = 2 π f & ( 1.5 ) \\ {R}_{2} = Re ( \frac{1}{{Y}_{22} + {Y}_{12}} ) & ( 1.6 ) \\ {C}_{2} = Im \frac{{Y}_{22} + {Y}_{12}}{ω} , where ω = 2 π f & ( 1.7 ) \\ R = Re ( Z ) , where Z = - \frac{1}{{Y}_{21}} & ( 1.8 ) \\ L = \frac{Im ( z )}{ω} , where Z = - \frac{1}{{Y}_{21}} and ω = 2 π f & ( 1.9 )\end{matrix}$$

As shown for selected parameters S₁₁ and S₁₂ in FIGS. 10A and 10B, when no sample is present between the antennas, strong resonances are seen wherein the transmission or reflection coefficients are particularly high at specific frequencies that vary periodically from about 0 to 800 MHz.

In comparison FIG. 10A and FIG. 10B also show parameters S₁₁ and S₂₂ when the test subjects finger is inserted in the probe region between the antennas. The resonance and spectral characteristics change dramatically due to the interaction with the molecule species in the tissue.

Although most of the cross variation in the spectral intensity of any of the Sij parameters is dominated by the antennas resonance pattern, it has been discovered through extensive statistical analysis that small portions of the spectra will correlate very well a patient's blood glucose concentrations.

It has also been discovered that more accurate and reproducible measurements of blood glucose can be obtaining using 4 antennas in a slightly different finger and hand mold, which is now illustrated in FIGS. 6A and 6B. The antennas supporting mold 301 has four external connectors 302 to the antennas.

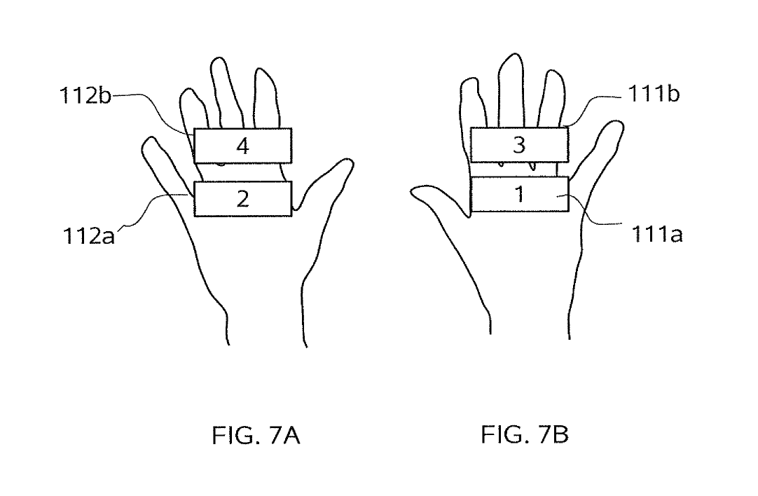

it was also discovered that further improvements were obtained when the antennas had a rectangular shape as shown in FIG. 8 and are oriented around the hand as shown in FIG. 7. It is thus currently preferred that the antenna 111/112 have an aspect ratio of 2:1. It should also be noted that superior results were obtained when the longer axis of the rectangular antenna is oriented perpendicular to the fingers, as shown in FIGS. 7A and 7B.

The antennas pairs 111a/112a and 111b/112b in FIGS. 8A and 8B have external dimensions of about 1 cm by 2 cm with the flat antenna coil having a width of about 75 μm being spaced apart from the adjacent winding by about 125 microns (for a 200 micron center to center spacing) to provide a total antennas length of about 130 cm, having about 20 to 25 turns. The individual antennas are simultaneously labeled 1(111a), 2(112a), 3(111b) and 4(112b) to comport with the mathematical treatments that follow. It should now be appreciated that the deployment of four antennas permitted the measurement and analysis of at least 10 basic S-parameters, that is 4 reflection coefficients (S₁₁, S₂₂, S₃₃, S₄₄) and 6 transmission coefficients (S₁₂, S₁₃, S₁₄, S₂₃, S₂₄, S₃₂, S₃₄). It should be understood that for reflection and transmission coefficient or parameter, the experimental amplitude and phase may be used together or separately in the data analysis and extraction that follow. It should also be noted that transmission coefficient S₁₃ and S₂₄ refer to adjacent antennas on the same side of the hand in which the electromagnetic radiation extends through the tissue, but is not transmitted perpendicular to the plane of the coiled antenna. In contrast, transmission coefficient S₁₂ and S₃₄refer to antennas on the direct opposite side of the hand in which the electromagnetic radiation extends through the tissue and is transmitted perpendicular to the plane of the coiled antenna. However, transmission coefficient S₁₄ and S₂₃ refer to antennas on the opposite side of the hand that are not directly opposite each other, in which the electromagnetic radiation extends through the tissue and is not transmitted perpendicular to the plane of the coiled antenna.

Not wishing to be bound by theory, it is currently believed that the resonances characteristics of the novel antenna designs have several distinct advantages over prior methods of dielectric spectroscopy to measure or estimate the in-vivo availability glucose in a patient. It is also believed that the antennas designs have distinct advantages in measuring glucose and other molecule in in-vitro.

Deploying antennas that generate deeply penetrating electromagnetic in the most desired range of about 100 to 800 MHz (0.1 to 0.8 GHz) provided more opportunity for the discovery of particular narrow frequency bands that gave good correlations with blood glucose and where also relatively insensitive to sources of error that have hindered the advance of earlier approaches to non-invasive measurements of blood glucose.

This was particularly the case when the resonant characteristics of antennas 111/112 are tuned for the media of interest such that the loss in transmission is generally less than −50 db, but more preferably less than about −30 dB between about 100 MHz and 800 MHz, but more preferably between about 1 MHz to 500 Mhz.

For detecting glucose in living tissue using the inventive method we have discovered it is preferable that the coiled antenna have a first resonance below about 100 MHz, but more preferable below about 50 MHz.

It has also been discovered that it is preferable that the coiled antennas also provide a characteristic zone of flat transmission coefficients in the media of interest over a range of about 200 MHz in which the transmission varies by less than about 30 dB, but more preferably less than about 20 dB, and the loss in transmission also less than −50 db, but more preferably less than about −30 dB. This range is typically up scale, higher wavelength that the first resonant frequency.

As the frequency of the first resonance of microwave antennas is inversely proportion to the antenna length, meeting this requirement posed a particular challenge to a conflicting need to make the antennas as small as possible for patient convenience and obtaining local measurements. However, both these requirements could be met by keeping the antennas width and spacing as narrow as possible and using multiple folds to obtain a long length, as for example the antenna 111 in FIG. 8 has at least 15 to 20 turns. This is in dramatic contrast to the prior art work in dielectric spectroscopy in which the antennas are in fact tuned to have a first resonance above 1 GHz to measure the shift in resonant frequency that is was observed by McClung (M S Thesis M. J. McClung, titled “Calibration Methodology for a Microwave Non-Invasive Glucose Sensor”, Baylor University, Department of Electrical and Computer Engineering 2008). The antennas disclosed therein has only 3 widely spaced turns, and the receiving antenna is a pair of strips placed on the same side of the thumb as this short coiled antenna. Therefore, the frequency shift measured by McClung is not actually in transmission, but is in fact a reflection coefficient.

The apparatus and method disclosed herein is expected to be more accurate than other methods of dielectric spectroscopy for several reasons. First, the prominent resonance peaks provide a stronger interaction with the dielectric relaxation properties of glucose and are less affected by the absorption from other molecules. This method thus appears to overcome electrode polarization effects noted in the prior art. Further, the inventive method is likely to be more representative of the bio-availability of glucose, as the measurement is more than skin deep.

Further, such deeper sampling of tissue by the inventive method is likely to produce more temporally stable results, being less sensitive to skin temperature and other skin conditions such as dirt, contamination and moisture and the like.

As the dielectric spectra is from a resonant system that will inherently vary with the electrode placement and physiology of each user or patient, it is not possible to precisely define universal lines or ranges of the spectra that are applicable to all patient's or test subjects.

However, it has been discovered that for each user/patient with a particular antenna combination disposed in signal communication across a particular body part or organ it is possible to identify the spectral ranges that correlate well with actual blood glucose concentration.

Accordingly, another aspect of the invention is a process for discovering such portions of the spectrum for each patient for use as a means of continuously and non-invasively accurately determining the blood glucose levels.

Yet another aspect of the invention are methods to develop the most robust means of continuously and non-invasively accurately determining the blood glucose levels.

It should be appreciated that such methods require the collection of data from a patient/subject equipped with the inventive antenna combination over a period that is sufficiently long to record a range actual blood glucose levels that is at least close to those likely to occur in real world conditions.

In the simplest mode of deployment such a device can warn the patient to better control dangerous excursions through the time administration of a source of glucose, generally by eating a healthy meal, or insulin.

In a more advanced mode of deployment such a device is anticipated to guide a patient to better control the blood glucose level within a narrower range to minimize the longer term and generally debilitating effects of diabetes, such as diabetic retinopathy, a proneness to infections and the like.

It is also anticipated that the potential for accurate and continuous measurement will enable integration into an artificial pancreas that in a closed feedback loop to a pump that can continuously provide insulin in response to the blood glucose levels.

According, the currently preferred methods of such embodiments are disclosed in the experimental description in the paragraphs that follow.

### Experimental Method and Results

Using the antennas and antennas supporting molds of FIG. 6-8, VNA spectra were collected in continuous mode using a commercial data acquisition system and program (LabView). Typically VNA spectra consist of 1600 measurement points spanned linearly from 1 MHz till 800 MHz. A four ports model VNA (such as for example from Agilent Technologies, Santa Clara, Calif.) can provide sixteen spectra S_{t,j}, {i,j=1, . . . 4} of which four spectra S_{i,j} corresponds to the reflection parameters, while the others twelve S_{i,j}, t≠j corresponds to the transmission parameters. Since S_{i,j} have have to be equal to S_{j,i} for reciprocal media such as human tissue, there are ten parameters altogether S_{i,j}, i≧j corresponding to the upper triangular matrix S_{i,j}.

The acquisition time of sixteen VNA spectra is about 30 sec. The SNR of the VNA in transmission mode is about −120 dB, while the signal level in transmission mode is in the range −30 dB to −70 dB, depending on frequency.

Each of VNA spectra S_{i,j} collected in continuous mode with a sample time τ can be organized into the N×M matrix

S_{i,j}(ωₘ, tₙ), {m=1,2, . . . M and n=1,2, . . . N} (2.0)

Where M=1600 is the number of frequency points and N is the number of collected spectra.

### Data Cross-Correlation Analysis

Fixing a frequency ωₖ in S_{i,j}(ωₖ,tₙ), we obtain a function of time fₖ(t)=S(ωₖ, t) (indexes i,j are omitted); thus we can consider a correlation function

p(k,l)=corr(fₖ(t), f(t)), k,l=1, . . . , M (2.1)

As follows from the Equation 2.1, ρ(k,l) is the matrix of size M×M with values of p(k,l) from −1 to 1.

### Correlelogram Analysis

Assume that we have a target function g(t) (such as glucose concentration) measured at same set of the sampling times tₖ, k=1,2, . . . K. This set can be a subset of the set of all times tₙ, n=1,2, . . . N.

From two functions g(t) and S_{i,j}(ω,t), we can build a correlation product over time.

rᵢⱼ(ω)=corr(S_{i,j}(ω,t), g(t)), k,l=1, . . . , M (2.2)

The correlation product is the correlation function of the measured reflection and transmission coefficients Sᵢⱼ to at least a portion of the measured blood glucose concentration. In the case of determining the concentration of other molecules of interest, the concentration of the other molecules would be used. The function rᵢⱼ(ω) reflects degree of similarity between the data S_{i,j}(ω,t) and the target function at given frequency. As definition (2.1) suggests, the module of the functions rᵢⱼ is less than or equal to one.

This correlation product when derived using the glucose values g(t) that vary widely, as can be obtained in an oral glucose tolerance test (OGTT), provides a means to identify spectral ranges in which the measured reflection or transmission coefficient S_{i,j} correlated highly with the actual glucose concentration.

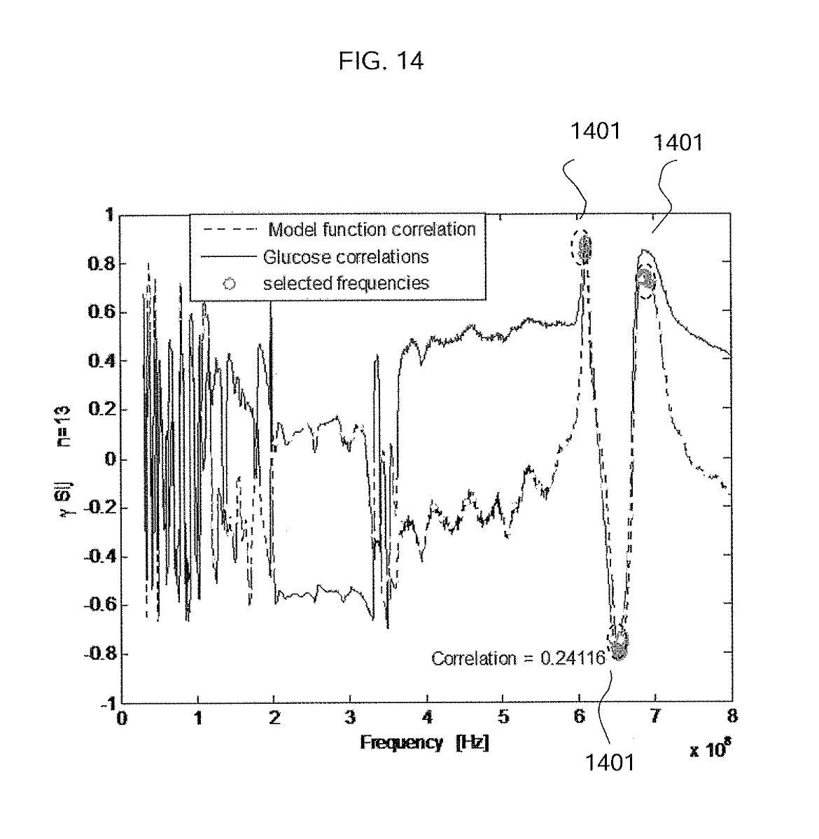

FIG. 14 represents a typical plot of the correlation product function r₂₄(ω). The solid curve in this figure shows correlation versus frequency with all the values of glucose concentration obtained during the OGTT, while the partially dashed curve shows the result of the same calculation using half the data. In this experiment this data was from before the large rise and fall of blood glucose induced by the OGTT. It is currently believed preferable to generate these curves from an OGTT using the broad peak in glucose as the partial data set, to determine if it will also predict the periods before and after when the blood glucose level are more stable. From definition (2.1) it follows that if S_{i,j} are the smooth functions of frequency, then rᵢⱼ are smooth functions as well. However, it is also believed possible to perform the above calculation using different combinations of partial data sets.

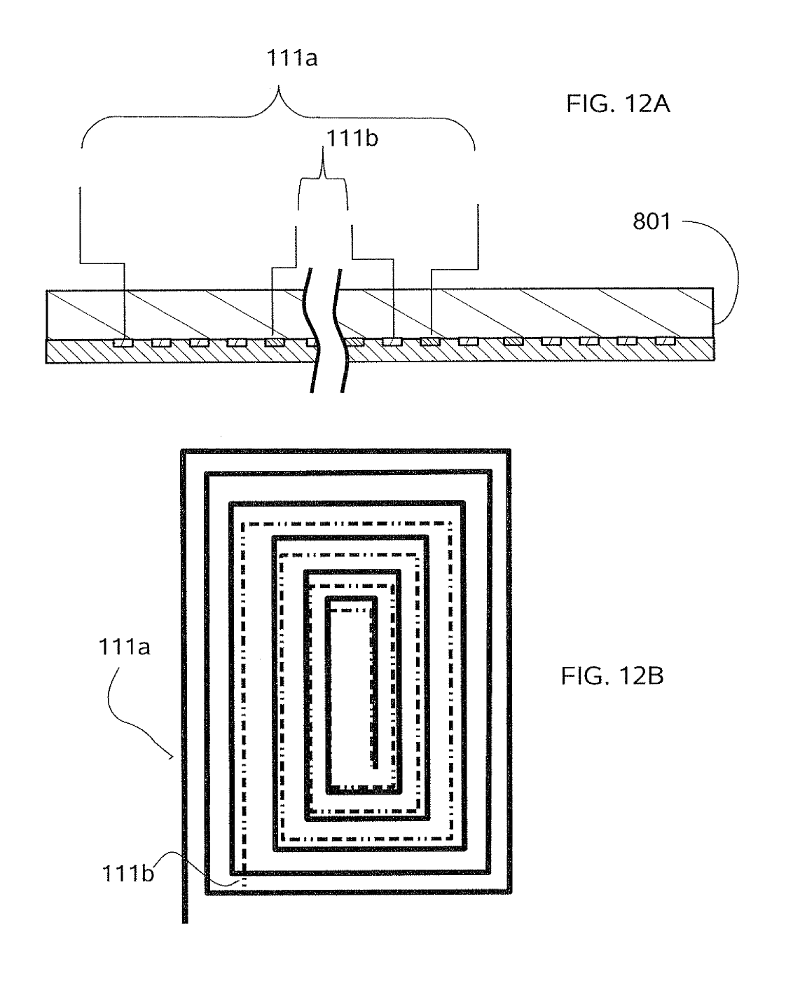

The example above shows that the behavior of the function r₂₄ (ω) is relatively smooth in the high frequency range of approximately from 350 MHz till 800 MHz, while the variation of this function is more considerable at low frequencies (below 200 MHz). By relatively smooth we mean a smaller ∂γij/∂(ω) than in the region of about 10 MHz to about 200 MHz where the narrow frequency bands corresponds to the antenna radiation pattern. It should now be appreciated that the most preferred antennas though having multiple resonances below 200 MHz frequency range, such that the response above 200 MHz is still relatively flat. FIG. 12 illustrates this desirable spectral response for S₁₂ for the antennas of FIG. 8 when deployed around the palm of the test subjects that is just below the fingers, as illustrated in FIGS. 7A and 7B.

It is more preferable to deploy the frequency range where the behavior of the functions rᵢⱼ is smooth to define the frequency or Q-bands, where the values of |rᵢⱼ| is more than some threshold value.

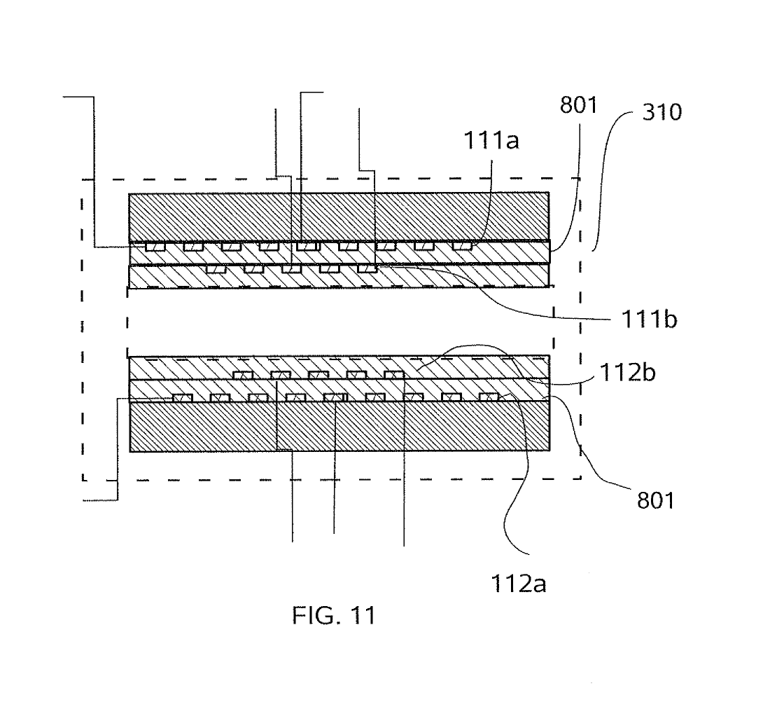

While experiments to date have used antennas of essentially identical sizes and patterns (as is limited by fabrication technology), when using either a total of 2 or 4 antennas, it may also be desirable to deploy 2 different pairs of 2 identical antennas, wherein the resonace characteristics of each antenna pair is different. Three different embodiments of this aspect of the invention are illustrated in. FIG. 11-13. This will allow the expansion of the optimum usefull spectral range, such as where the transmission is relatively flat and sufficiently high, where one pair of the antennas is optimized for a first spectral range that has at least a portion below higher optimized spectral range of the other antenna pair. The antenna pairs can occupy the same area by changing the spacing on one antennas coil, keeping the line width identical or varying in different portions. In such a case, it may also be preferable to provide some overlap in a spectral range where usefull information can be obtained for an additional cross-correlation or selection of the Q-bands.

Different pairs of antennas can be arranged in several ways in addition to configuration illustrated in FIG. 7. For example antennas 111a and 111b in the top half of antenna supporting mold 301 are superimposed laterally as shown in FIG. 11, but spaced apart slightly on different side of a portions of a PCB or flexible carrier tape 801, in which the spacing is the tape or PCB thickness, as are the other antennas of the pair 112a and 112b. Thus, antennas in pair 111a/112a can be longer to have a shorter or lower first resonance frequency while the antennas in pair 111b/112b can be shorter to have longer or larger first resonance frequency. The difference in spacing between antennas pairs 111a/112a and 111b/112b through the sample or tissue is twice the thickness of the PCB or flexible carrier tape 801.

Alternatively, as shown in FIGS. 12A and 12B, antenna pairs 111a/112a and 111b/112b can also have the same spacing through the sample or tissue by providing adjacent wrapped conductive traces on the same board or tape 801, with the shorter antenna 111b terminating first and having an external contact (shown in FIG. 11-13 as vertical lines) through a via in the PCB or tape 801. The shorter antennas 111b in the plan view in FIG. 12B is shown in a dashed line.

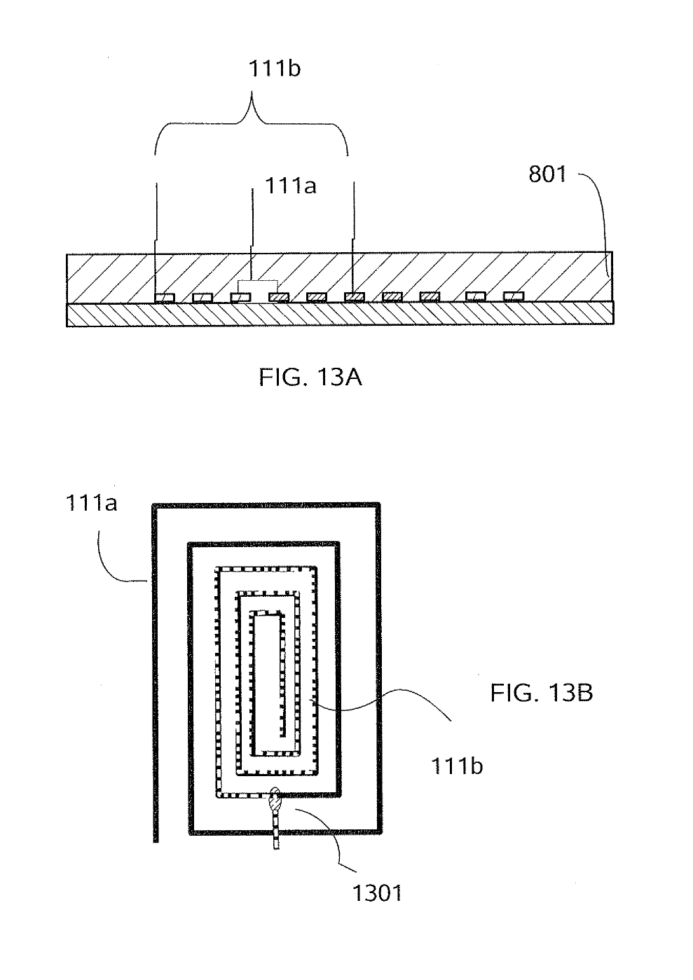

In the embodiment shown in FIG. 13, Antenna 111a is longer, as it uses a portion of the inner and shorter antenna 111b via a switch 1301, so there is only one spiral trace with the switch 1301 being in the spiral trace.

In addition to expanding the usefull spectral range, having such overlapping plural antenna pairs also provide a different penetration depth in the tissue for each pair to permit a continous comparison of the both glucose in tissue closer to the skin against what might be much deeper venous and arterial tissue. As the glucose in tissue closer to the skin is more likely to represent intersticial tissue, this may provide greater predictability of trends in glucose in the patient/test subject, as well as for greater accuracy of measurement.

Thus, after the acquisition of the different sets of signal propagation parameters Sᵢⱼ, the entire calibration process can be carried out fully automatically by a microprocessor or other computing means by first acquiring the data, that is S_{i,j}(ωₘ, tₙ), then calculating at least 2 sets of rᵢⱼ via the equation below using a complete and partial set of independently measured blood glusoce values. Further, the comparison of of these at least two sets can be an automated process as described below.

### Extracting the Q-Bands

The final predictive equation for blood glucose concentration requires the identification of frequency interval or bands of the spectral response of any Sij parameter in which model function and the measured glucose concentration are well correlated. This can be expressed mathematically as the set of all frequencies bands [ωₖ, ωₜ], l>k such that the inequality (2.3) holds are called Q-bands.

∀ ω [symbol] [ωₖ, ωₜ], [rᵢⱼ(ω)|≧c (2.3)

Where c is a threshold value. That is, a set of Q-bands are selected where absolute value of rᵢⱼ is greater than or equal to a threshold value, C, from some band width represented by ωₖ to ωᵢ. This correlation threshold, C, is preferably at least about 0.75. Ideally such Q-bands should not overlap with each other. Thus, within each Q-band the correlation of S_{i,j} and the target function g(t) is more than the threshold value. FIG. 14 shows example of three Q-bands where the correlation with the target function (partial glucose data) and S₂₄ are more than 0.75, as highlighted by the broken circles 1401.

For each Q-band [ωₖ,ωₗ]ᵢⱼ (indexes i,j here corresponds to the indexes of the S_{i,j} one can extract a feature function by averaging S_{i,j}(ω,t) over the interval [ωₖ, ωₗ].

f_{kl,ij}(t)= [symbol] S_{i,j}(ω,t) [symbol] (2.5)

The definition (2.3) insures that correlating of the f_{kl,ij}(t) and the target function g(t) will be not less that the threshold value c.

The above equations thus provide an algorithm for generating feature functions from the set of Q-bands that are highly correlated with blood glucose concentration of the patient, g(t).

Thus, a preferred mode of using the dual antenna apparatus 100 is to perform the previously described set of calculations on each patient during an initial OGTT, or similar diagnostic procedure that provides an opportunity to collect spectral data during a reasonably large excursion in blood glucose concentration when the actual glucose concentration is known very accurately by an independent method. This provides a set of candidate Sᵢⱼ parameters, each at one or more selected. Q-bands, to derive a predictive formula for calculating the patient blood glucose concentration continuously. Such sets may range from 10 to 30 potential Q-bands. The analysis to date of about a dozen individuals has revealed a general trend of identifying about 1 to 4 Q-bands for 7 to 10 of the Sᵢⱼ parameters.

A final predictive equation can be derived from the feature functions of equation (2.5) by a wide variety of known regression techniques for each of the feature functions, which are found by integrating the value of the Q-band parameters selected as candidates in the previous set of 10 to 30 Q-bands.

The correlation coefficient for each of the feature function corresponding to specific Sij parameters over the Q-band frequency ranges can then be compared so that only the most highly correlated feature functions are used in the final predictive equation. However, it has also been found preferable to use additional criteria for selecting a limited set of Sᵢⱼ parameters to derive and select the feature functions used in the final predictive formula. Among these criteria it is preferred to compare the temporal stability of the Q-band over a time period when the blood glucose is quite stable. Thus, in this case rather than scanning over the entire band width used to discover Q-bands, just the narrow Q-band would be repeatedly scanned. Such scans can be much faster than 30 seconds, and can be repeated as needed to compare their temporal stability as well as the signal to noise ratio. In this manner, the Q-bands used to derive the final predictive equation can be selected based on their having the highest signal to noise ratio.

It is additionally preferable to also or alternatively select the Sᵢⱼ/Q-band parameters that are relatively insensitive to external effects, for example temperature and well as precise positioning in the antenna holder 301. The exploration of a correlation with temperature is easily performed for each Q-band if there is sufficient temperature excursion either during or after the initial data collection step when the device 100 also includes a thermocouple of non-contact IR thermometer.

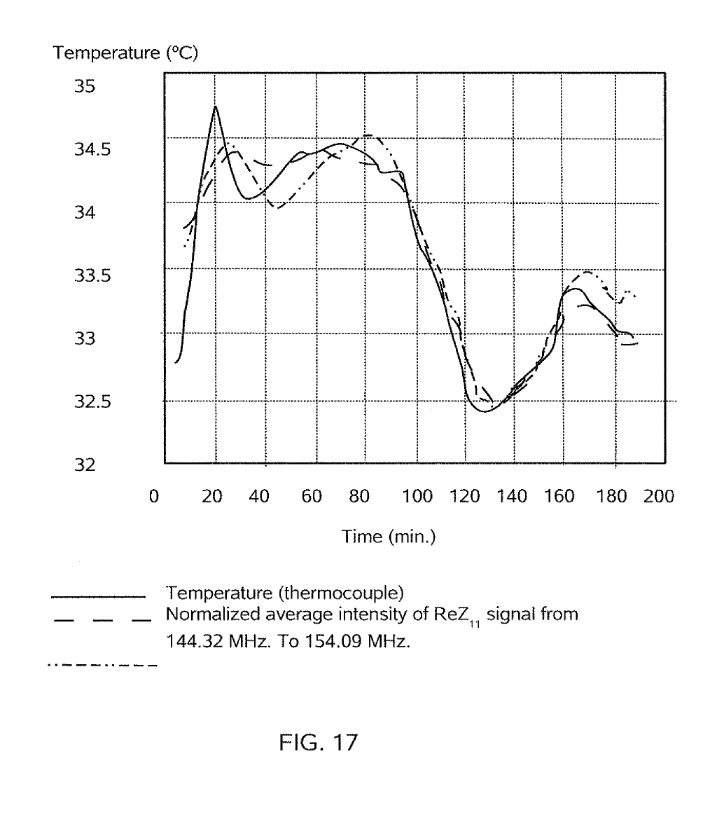

FIG. 17 illustrates an observed correlations of temperature dependence of the integrated intensity of selected model circuit parameters, integrated over specific narrow frequency ranges showing a strong correlation with temperature without a strong dependence on blood glucose. As opposed to prior art methods of dielectric spectroscopy based glucometry, if it should be necessary to empirically correct for temperature variation, it is likely that the need for temperature sensors to estimate the actual temperature of the sampled skin depth can be avoided, as the measurement system itself provides a means to measure the actual temperature of the tissue within the depth being sampled. Thus, inventive method is likely to provide an improved means to calibrate and correct for temperature variation of the subject and the environmental effects thereon.

As to the sensitivity of candidate Q-band to position of the holder device on the patient/test subject, it has been discovered that more reproducible results are obtained by first acquiring the spectra over the candidate Q-bands repeated times in sequence and then calculating the standard deviation of each Sij value as integrated over the Q-band width to select Q-bands of lower standard deviation.

Ideally, a limited selection of all possible Sij and associated band regions parameters are selected for regression analysis. While various forms of Chemometrics techniques for multivariable regression can be performed on a plurality of Sᵢⱼ parameters, as the objective of the present invention is to provide a diagnostic tool, it is currently preferred that a single Sᵢⱼ parameter be derived by linear regression that provides a good fit to the measured glucose values in the ranges of clinical importance. Thus, another criteria for selecting the most appropriate Q-band is based on the lowest error in the regression analysis.

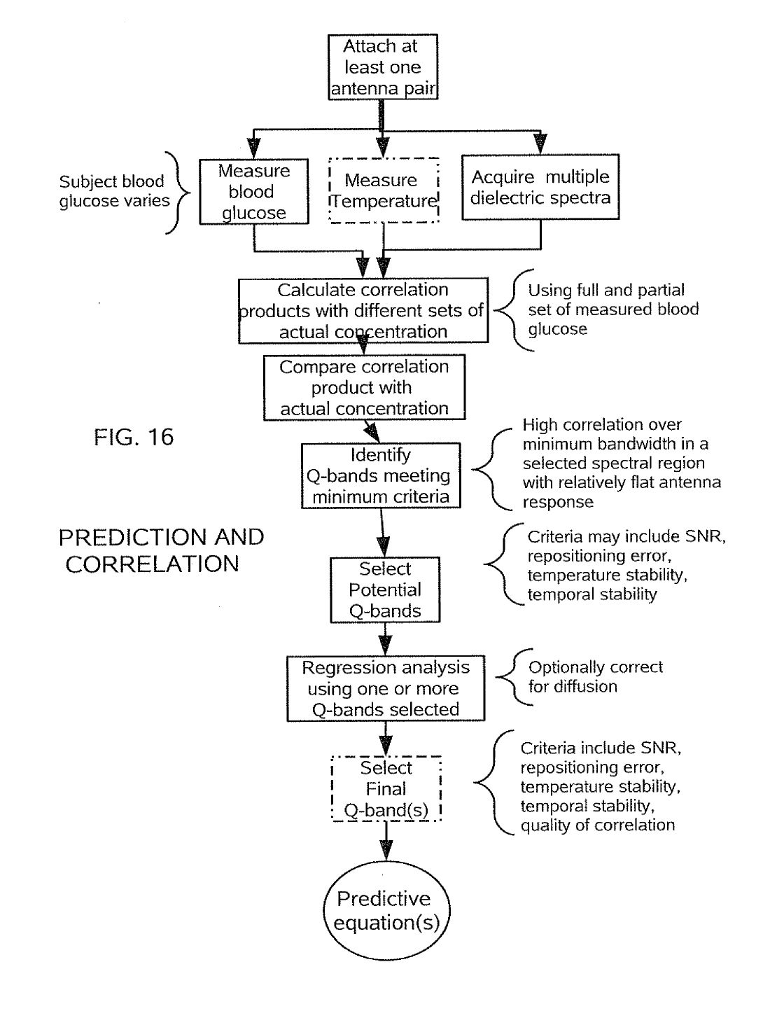

The flow chart in FIG. 16 summarizes the above measurement and calibration steps, including the other criteria for Q-band selection described more fully below.

Another aspect of the calibration process is to select the optimum Sᵢⱼ parameter that correlates best and most robustly with the measured blood glucose concentration as measured by convention methods in either the hospital or clinical setting, or those routinely used by diabetic patients.

Part of such optimization is insuring that particular parameter is robust with respect to a minimum noise and errors that occur repeated removal and insertion of the hand in the antenna supporting mold 301, or alternatively with respect to any other fixture that holds and support the pair of at least 2 antennas if deployed to measure blood glucose on another organ or part of the body than the hand.

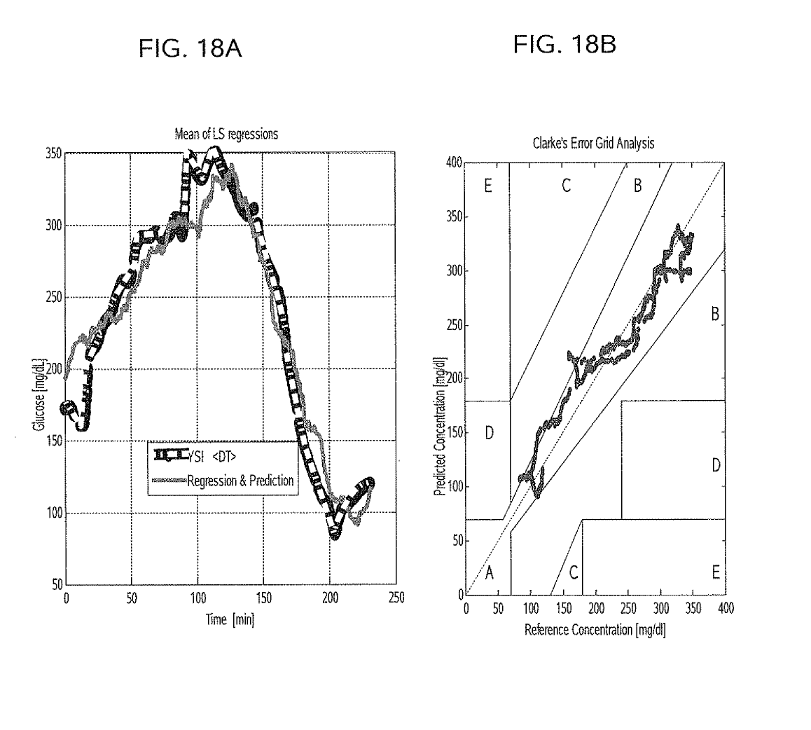

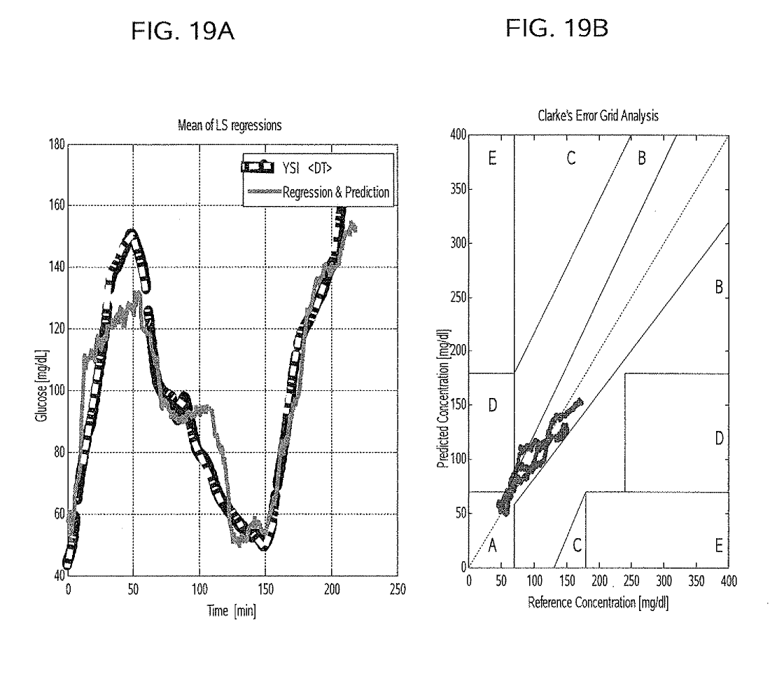

Clinical trials have been conducted using the techniques described above. The predicted blood glucose level from the trial is compared in the Clark grid in FIGS. 18 and 19. The most productive Q-bands identified in a relatively small subset of patients/subjects are listed in Tables 1 and 2 below. In these tables the first column identifies the signal parameter, which is intensity, when not referred to specifically with the subscript “ph” for phase. The second column is the channel count range for this band, while the third column is the equivalent frequency range in MHz, for the Q-band. The fourth column is the correlation coefficient with the actual blood glucose measurement, as made by the YSI method. Table 1 contains an extra column between the fifth and last column showing the standard deviation in re-positioning error. The fifth column is the correlation coefficient with temperature. The column to the farthest right in the table is the signal to noise ratio of the Q-band that was calculated as the STD. of the reposition error divided by the signal amplitude.

Table 1 refers to tests taken when the subjects were subjected to an OGTT to produce a hyperglycemic state, with the glucose concentration ranging from about 100 to 350 mg/dl. Table 2 refers to tests taken when the subjects were administered a very controlled dose of insulin to lower the insulin levels to the hypoglycemic state, with the blood glucose levels ranging from 50 to 175 mg/dl. The predicative result of the 10 “best” Q-bands in the table were then averaged after linear (uni-variant) regression to provide the final linear predictive equation as described above, and are plotted as the solid line “Regression and Prediction” against the blood glucose measured by YSI , which is the wider partially broken line.

TABLE I

| | | | CC Calibration | | | |
|---|---|---|---|---|---|---|
| | | | vs | CC Calibration vs | Deviation | Signal |
| Type | Position | Frequency | Glucose | Temperature | Reposition | Noise |
| S11db | [263, 268] | [56.17, 158.57] MHz | -91.0066 | -55.7116 | stdr = 1.3 | 3.783294 |
| S12 | [959, 969] | [491.33, 496.14] MHz | -92.6765 | -58.7426 | stdr = 5.1 | 5.118336 |
| S12 | [1061, 1114] | [540.44, 565.97] MHz | -91.9049 | -63.3541 | stdr = 3.1 | 2.688731 |
| S12 | [1221, 1263] | [617.49, 637.72] MHz | -91.1643 | -67.4358 | stdr = 5 | 3.150891 |
| S13 | [146, 146] | [99.82, 99.82] MHz | 92.72564 | 59.04039 | stdr = 2 | 3.968204 |
| S44db | [661, 661] | [347.82, 347.82] MHz | -92.147 | -67.2053 | stdr = 3.2 | 5.384147 |
| S14ph | [3, 3] | [30.96, 30.96] MHz | 91.26106 | 74.28393 | stdr = 15430.9 | 5.145149 |
| S14ph | [146, 146] | [99.82, 99.82] MHz | 93.05493 | 60.93371 | stdr = 65883.6 | 4.360244 |
| S22ph | [854, 858] | [440.76, 442.69] MHz | 91.16346 | 74.64618 | stdr = 22 | 4.178983 |
| S23ph | [1518, 1537] | [760.51, 769.66] MHz | 93.21846 | 71.18909 | stdr = 1058970.7 | 2.982021 |
| S34ph | [679, 685] | [356.49, 359.38] MHz | 92.97443 | 71.50172 | stdr = 5806134.1 | 2.594565 |
| S34ph | [142, 145] | [97.9, 99.82] MHz | -95.6516 | -58.746 | stdr = 246575.3 | 3.933949 |
| S44ph | [1287, 1288] | [649.27, 649.76] MHz | -91.7352 | -62.9503 | stdr = 11.7 | 7.108362 |

The average of the data from the Q-bands in the above Table 1 is plotted against the actual glucose concentration in FIG. 18A, of which the corresponding Clark grid is shown in FIG. 18B for a group of patients.

TABLE 2

| | | | CC Calibration | CC Calibration vs | Signal |
|---|---|---|---|---|---|
| Type | Position | Frequency | vs Glucose | Temperature | Noise |
| S11db | [662, 664] | [348.31, 349.27] MHz | 85.10891 | 52.71584 | 5.633072 |
| S13 | [251, 279] | [150.39, 163.87] MHz | 85.00375 | 44.30454 | 4.421911 |
| S22db | [791, 800] | [410.43, 414.76] MHz | 89.91409 | 55.29072 | 5.216331 |
| S22db | [1284, 1284] | [647.83, 647.83] MHz | −85.4515 | −21.6692 | 4.059745 |
| S13ph | [248, 254] | [148.94, 151.83] MHz | −86.5356 | −55.13 | 5.396657 |
| S22ph | [316, 316] | [181.69, 181.69] MHz | −85.8894 | −54.4095 | 4.201929 |
| S24ph | [121, 127] | [87.79, 90.68] MHz | 89.9217 | 55.68879 | 4.28766 |
| S24ph | [401, 406] | [222.62, 225.03] MHz | 87.39871 | 41.29046 | 4.411985 |

The average of the data from the Q-bands in the above Table 2 is plotted against the actual glucose concentration in FIG. 19A of which the corresponding Clark grid is shown in FIG. 19B.

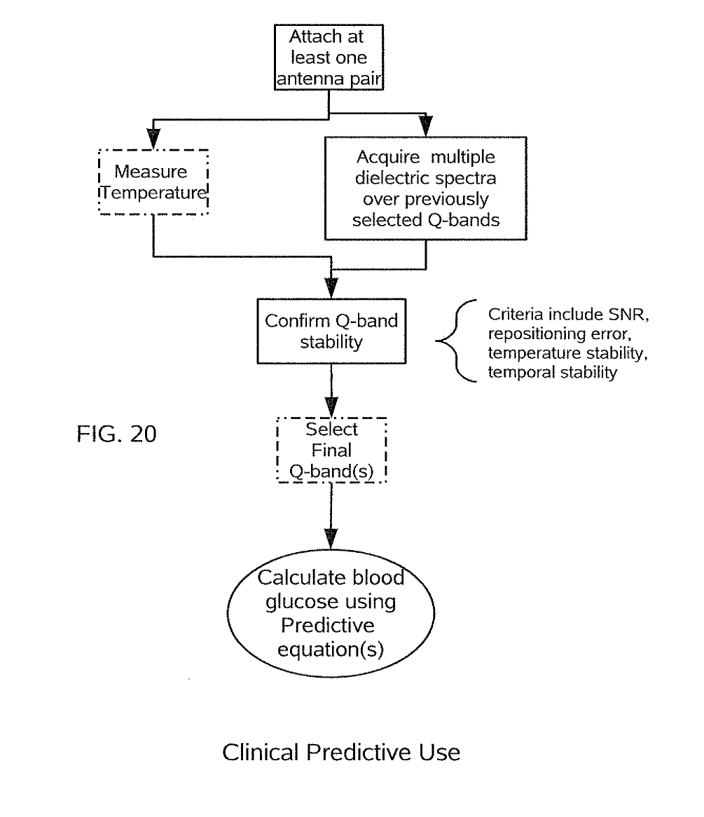

FIG. 20 is flowchart illustrating the steps of using the device disclosed herein to monitor blood glucose to non-invasively and continuous after the steps if calibration of FIG. 16.

While the invention has been described in connection with a preferred embodiment, it is not intended to limit the scope of the invention to the particular form set forth, but on the contrary, it is intended to cover such alternatives, modifications, and equivalents as may be within the spirit and scope of the invention as defined by the appended claims.

1) A process for molecular spectroscopy of a media to determine the concentration of at least one molecular species therein, the process comprising the steps of:
a) providing a pair of coiled antennas as electrodes for dielectric spectroscopy measurements,
b) placing the pair of coiled antenna in signal communication through the media,
c) powering at least one of coiled antennas at a first frequency,
d) scanning a frequency range during said step of powering from the first frequency to at least a second frequency, the difference between the first and second frequency representing a first frequency range,
e) acquiring one or more signals from at least one of the coiled antennas during said step of scanning to determine the value thereof,
f) integrating the value of the one or more signals in said step of acquiring, the integration occurring over at least a portion of the first frequency range,
g) calculating the concentration of the molecular species from the integrated value of the one or more signals.

2) A process for molecular spectroscopy of a media to determine the concentration of at least one molecular species therein according to claim 1 wherein the media is living tissue and the coiled antennas have a first resonance below the second frequency range.

3) A process for molecular spectroscopy of a media to determine the concentration of at least one molecular species therein according to claim 1 wherein the coiled antennas interact with media over a second frequency range having a width of at least about 200 MHz in which the transmission varies by less than about 30 dB and the transmission loss is less than about −50 db and the first frequency range includes at least a portion of the second frequency range.

4) A process for molecular spectroscopy of a media to determine the concentration of at least one molecular species therein according to claim 3 wherein the media is living tissue and the coiled antennas have a first resonance below about 100 MHz.

5) A process for molecular spectroscopy of a media to determine the concentration of at least one molecular species therein according to claim 4 wherein the molecular species is glucose.

6) A process for molecular spectroscopy of a media to determine the concentration of at least one molecular species therein according to claim 5 in which the transmission loss in the second frequency range is less than about −30 db.

7) A device for the in-vivo molecular spectroscopy, the device comprising:
a) at least one pair of coiled antennas and configured for placement in signal communication with the other antennas in the pair through a first dielectric medium comprising at least a portion of a living organism,
b) a variable frequency power generator in signal communication to each of the antennas in said pair,
c) a signal detector in communication to each of the antennas in said pair for collecting transmitted and reflected signals between each of the antennas over the generated frequency range,
d) a computation means to determine a plurality of signal propagation constants from the detected signals and calculate the concentration of at least one molecular species there from, wherein the pair of coiled antennas have a first resonance below about 100 MHz and the concentration of the molecular species is calculated by integration of one or more of the plurality of signal propagation constants over a frequency range from a first lower frequency to a second upper frequency wherein the second upper frequency is less than about 1 GHz.

8) A device for the in-vivo molecular spectroscopy according to claim 7 wherein the antennas in the first pair are adjacent to each other on the same side of the organism.

9) A device for the in-vivo molecular spectroscopy according to claim 7 further comprising an enclosure for supporting each of the antennas in the pair on opposite sides of a portion of the living organism between a gap for receiving the portion of the living organism, the enclosure having an opposing first and second face in a spaced apart relationship, wherein the first and second face are substantially perpendicular to each other, and;
a) the first antenna of the pair has a coiled conductive path substantially disposed in a first common plane and supported by the enclosure wherein the first common plane is disposed in spaced apart relationship behind the first face of the enclosure by a first distance so as to be immersed in a second dielectric medium,
b) the second antenna of the pair has a coiled conductive path substantially disposed in a second common plane and supported by the enclosure wherein the second common plane is disposed in spaced apart relationship behind the second face of the enclosure by a second distance so as to be immersed in a second dielectric medium.

10) A device for the in-vivo molecular spectroscopy according to claim 7 further comprising a second pair of coiled antennas, the second pair of coiled antennas being configured for placement in signal communication with the other antennas in the pair through a first dielectric medium comprising at least a portion of a living organism wherein the a variable frequency power generator is in signal communication to each of the antennas in each pair.

11) A device for the in-vivo molecular spectroscopy according to claim 10 where the first and second pairs of antennas are adjacent.

12) A device for the in-vivo molecular spectroscopy according to claim 10 wherein the first and second pairs of antennas have a different first resonance frequency.

13) A device for the in-vivo molecular spectroscopy according to claim 10 where the first and second pairs of antennas overlap to sample at least an overlapping portion of the living organism.

14) A device for the in-vivo molecular spectroscopy according to claim 10 wherein one of the first and second pairs of antenna is coiled within the other pair, being disposed substantially within the same plane thereof.

15) A device for the in-vivo molecular spectroscopy according to claim 10 wherein the second dielectric medium has a thickness of at least 300 μm to about 5 mm.

16) A process to calibrate a device for molecular spectroscopy of a media to determine the concentration of at least one molecular species therein, the process comprising the steps of:
a) providing at least one sample media through which a plurality of different concentrations of the molecular species is at least one of known and determinable by independent means of the molecular spectroscopy process,
b) providing a pair of coiled antennas as electrodes for dielectric spectroscopy measurements,
c) placing the pair of coiled antennas in signal communication through the sample media,
d) powering at least one of coiled antennas at a first frequency,
e) scanning a frequency range during said step of powering from the first frequency to at least a second frequency, the difference between the first and second frequency representing a first frequency range,
f) repeating said step of scanning of the sample media at plurality of times each corresponding to the different concentrations of the molecular species that is at least one of known and determinable by independent means of the molecular spectroscopy process,
g) acquiring one or more signals from at least one of the coiled antennas during said steps of repeated scanning to determine the value of a plurality of signal propagation parameters,
h) calculating a first correlation product of each of the signal propagation parameters with at least a first subset of the known or determined concentrations of the molecular species,
i) calculating at second correlation product of each of the signal propagation parameters with at least a second subset of the known or determined concentrations of the molecular species, the second subset being larger than the first subset,
j) comparing the first and second correlation products over the first frequency range,
k) identify at least one signal propagation parameter having a selecting regions within the first frequency range wherein the absolute value of the correlation product is greater than about 0.75 over a continuous second frequency range having a width of at least about 50 MHz,
l) calculating the integrated value of each signal propagation parameter identified in the previous step over the continuous second frequency associated therewith with provide at least one Q-band parameters,
m) calculating the correlation of the at least one Q-band parameter to the known or determined concentrations of the molecular species to provide a calibration equation.

17) A process to calibrate a device for molecular spectroscopy of a media to determine the concentration of at least one molecular species therein according to claim 16 and further comprising the steps of:
a) acquiring temperature of the media during said steps of repeated scanning to determine the value of a plurality of signal propagation parameters.

18) A process to calibrate a device for molecular spectroscopy of a media to determine the concentration of at least one molecular species therein according to claim 16 and further comprising the steps of:
a) acquiring a plurality Q-band parameters
b) characterizing each of the Q-band parameters in the plurality by at least one of;
i) SNR,
ii) repositioning error,
iii) temperature stability,
iv) temporal stability,
v) quality of correlation to the known or determined concentration of the molecular species,
c) selecting from the plurality of characterized Q-band parameters a smaller subset based on the characterization thereof in the previous step,
d) wherein said calculating the correlation of the at least one Q-band parameter to the known or determined concentrations of the molecular species to provide a calibration equation uses a Q-band parameter selected from the smaller subset.

19) A process to calibrate a device for molecular spectroscopy of a media to determine the concentration of at least one molecular species therein according to claim 16 wherein the media is at least one of fluid and living tissue and the molecular species is glucose.

20) A process to calibrate a device for molecular spectroscopy of a media to determine the concentration of at least one molecular species therein according to claim 15 wherein;
a) said step of providing at least one sample media through which a plurality of different concentrations of the molecular species is at least one of known and determinable by independent means of the molecular spectroscopy further comprises providing a plurality of molecular species at a plurality of different concentrations, and
b) said step of calculating the integrated value of each signal propagation parameter identified in the previous step over the continuous second frequency associated therewith provides at two or more Q-band parameters, each of which correlates with a different molecular species.
